(19) United States
(12) Patent Application Publication
(10) Pub. No.: US 2017/0229041 A1
(43) Pub. Date: Aug. 10, 2017

(54) COORDINATED PHYSICAL AND SENSORY TRAINING

(71) Applicants: Alan REICHOW, Falls, IA (US); Stephen SWANSON, Park City, UT (US)

(72) Inventors: Alan Reichow, Cedar Falls, IA (US); Stephen Swanson, Park City, UT (US)

(73) Assignee: Instinct Performance LLC, Oklahoma City, OK (US)

(21) Appl. No.: 15/502,470

(22) PCT Filed: Aug. 7, 2015
(86) PCT No.: PCT/US15/44124

§ 371 Date: Feb. 7, 2017

Related U.S. Application Data
(60) Provisional application No. 62/034,244, filed on Aug. 7, 2014.
(60) Provisional application No. 62/065,263, filed on Oct. 17, 2014.
(60) Provisional application No. 62/086,489, filed on Dec. 2, 2014.

(51) Int. Cl. G09B 21/00; A61B 5/11; G09B 19/00; G09B 9/00; G09B 9/052; A61B 5/00; A63B 69/00

(52) U.S. Cl. CPC G09B 21/008; A61B 5/4023; A61B 5/742; A61B 5/486; A61B 5/6807; A61B 5/11; A61B 5/6804; A61B 5/6895; A63B 69/00; G09B 9/006; G09B 9/052; G09B 19/0038; A61B 2562/0219; A61B 2562/0247

(57) ABSTRACT

Physical and/or sensory skills may be trained by varying the quantity of sensory information available to an individual, the quality of sensory information available to an individual, and/or the difficulty of physical training performed by an individual. The difficulty of training may be varied using eyewear that alters the quality/quantity of visual information available and/or that provide a display that instructs the individual training to increase/decrease the difficulty of training tasks. The difficulty of training may be varied in response to measurements of the physiology and/or performance of an individual training.

### CROSS-REFERENCE TO RELATED APPLICATIONS

This application claims the benefit of provisional patent application Ser. No. 62/034,244, entitled “Coordinated Athletic and Sensory Training,” filed on Aug. 7, 2014, which is incorporated herein by reference. This application also claims the benefit of provisional patent application Ser. No. 62/065,263, entitled “Perceptual Stress Training Eyewear Providing Recovery Periods,” filed on Oct. 7, 2014, which is incorporated herein by reference. This application also claims the benefit of provisional patent application Ser. No. 62/086,489, entitled “Wearable Sensors with Heads-up Display,” filed on Dec. 2, 2014, which is incorporated herein by reference.

### FIELD OF INVENTION

The present invention relates to systems and methods for training an individual's physical and sensory skills and abilities. More particularly, the present invention relates to systems and methods that combine sensory and physical training tasks.

### BACKGROUND AND DESCRIPTION OF THE RELATED ART

Typical day-to-day life requires a person to rely upon both sensory and physical abilities, typically in conjunction with one another. Competitive athletes may place greater demands upon their physical and sensory abilities than other individuals, but all individuals rely upon both sensory and physical abilities.

Successful athletes often possess innate physical abilities exceeding those of others, but mere physical ability, such as strength, speed, dexterity, and agility, is not usually enough to compete successfully at the highest level of a sport. Successful individuals must devote substantial time to training in order to improve their innate physical abilities and to develop specific skills needed to win in competition. Even non-athletes may engage in physical training for health benefits or simple pleasure. In some instances, however, individuals may engage in training to attempt to regain some or all of the abilities lost due to injury and/or illness.

### SUMMARY OF THE INVENTION

While physical skills and abilities have traditionally been improved by training, physical skill and ability exist in combination with sensory skills and abilities, a co-existence that is true across all ranges of abilities and all activities. Systems and methods in accordance with the present invention enable an individual to train his or her physical skills and abilities while also training his or her sensory skills and abilities. By varying the sensory challenge presented to the individual while the physical training tasks are performed, and/or by varying the challenge of the physical training tasks while a sensory challenge is presented, both sensory and physical skills may be improved. Further, the improvement of sensory skills within a context of desired physical performance can improve sensory performance within the context of that physical performance when the sensory training load is no longer present, such as at competition. Because physical skills are closely related to sensory skills, both may often be improved simultaneously though appropriate training.

While every individual uses both physical and sensory skills in tandem during the course of daily life, and may therefore benefit from systems and methods in accordance with the present invention, training of sensory and physical skills may be particularly beneficial for individuals such as rehabilitation patients, who may be seeking to re-attain their prior sensory and physical skills, and athletes, who may be seeking a competitive advantage through improved sensory and physical skills.

Sensory abilities and skills are a component of athletic success. For example, no amount of deft touch with her foot will permit a soccer player to excel if she is unable to accurately judge the trajectory of an incoming ball. While different sports may require different visual or other sensory skills, virtually all athletes, regardless of sport, may improve their performance by enhancing their sensory abilities and by developing specific sensory skills beneficial to their competitions. For athletes, sensory skills do not typically exist in isolation. For example, a baseball player may wish to maximize his visual abilities not to attain an impressive performance on a vision examination but to better identify a pitch type, speed, and anticipated time and location of contact while batting during a game. By incorporating sensory training into athletic training, systems and methods in accordance with the present invention improve both athletic and sensory abilities/skills, while also developing sensory abilities/skills within the context of the athlete's sport.

For many athletes, the interconnection between their vision and their balance and stability is critical to competitive success. For example, in shooting sports an athlete must, of course accurately judge the location of the target and aim correctly, but in order to consistently hit the target the athlete must reliably maintain stable balance while shooting. The demands of maintaining balance and stability while visually tracking a target can be even greater for competitions such as trap shooting, where the target is moving. For such an athlete to reliably maintain his or her balance while visually acquiring the moving target places considerable demands on both the physical skills of the athlete and the sensory skills of the athlete, and those demands only grow as the athlete must quickly aim, track, and fire. A similar challenge to the stability of an athlete arises in the game of golf. In golf, even though the ball to be struck is stationary, an athlete holds his or her head at a downward angle and then rotates his or her body and neck while the eyes remain visually locked on the ball, dynamics that challenge the golfer's balance. The criticality of balance may be even more acute in rehabilitation scenarios, where increasing or restoring the ability to maintain one's equilibrium while performing physical tasks reliant upon visual inputs may be a key step to an improved quality of life.

Individuals seeking to improve their sensory abilities and associated physical performance abilities may be suffering from impairments, such as may be due to traumatic head injuries, stroke, or other illness or injury. For such individuals, improved abilities to integrate sensory data may greatly improve their quality of life. For example, an individual with impaired balance, such as may be caused by traumatic head injuries, strokes, and other causes, may benefit from training to better integrate visual data with other senses to better walk, stand, and/or interact with their environment. On the other hand, even individuals with relatively strong sensory skills may benefit from sensory training in order to improve a physical performance at least partially dependent upon those sensory abilities. For example, individuals and other individuals engaged in vocations and/or avocations with outcomes dependent in some way upon successfully interacting with the perceived environment may find their performance improved by engaging in sensory and/or physical training, even if the individual would not normally be considered an “athlete” or to be engaged in any type of rehabilitation.

Systems and methods in accordance with the present invention may measure a physical/physiological characteristic of an individual training using one or more sensor. Such physiological measurements may be used to adjust the difficulty of the sensory and/or physical training to maintain a challenging but not overwhelming difficulty level. Physiological metrics may additionally/alternatively be provided to the individual training to provide guidance, a history of improvement, etc. One particularly valuable metric may be an indication of the balance or stability of an individual, but metrics such as heart rate, blood pressure, and/or eye movement may additionally/alternatively be used. Instead of or in addition to a physical/physiological measurements, systems and methods in accordance with the present invention may measure the results of a physical training task, and those results may similarly be provided to the individual training and/or used to adjust the difficulty of one or both of the sensory challenge and the physical training tasks.

### BRIEF DESCRIPTION OF THE SEVERAL VIEWS OF THE DRAWINGS

Examples of systems and methods in accordance with the present invention are described in conjunction with the attached drawings, wherein:

FIG. 1 schematically illustrates an exemplary system in accordance with the present invention;

FIG. 2 illustrates an example of an individual training using exemplary systems and methods in accordance with the present invention;

FIG. 3 schematically illustrates a further exemplary system in accordance with the present invention;

FIG. 4 illustrates an exemplary method in accordance with the present invention;

FIG. 5 illustrates a further example of an individual training using exemplary systems and methods in accordance with the present invention;

FIG. 6 illustrates and example of eyewear in accordance with the present invention;

FIGS. 7A-7D illustrate example indicia that may be displayed to an individual training using systems and methods in accordance with the present invention;

FIG. 8 illustrates a further example of a system in accordance with the present invention;

FIG. 9 illustrates a further exemplary method in accordance with the present invention;

FIG. 10 illustrates an additional example of a system in accordance with the present invention; and

FIG. 11 illustrates a further example of a method in accordance with the present invention.

### DETAILED DESCRIPTION

Sensory skills of an individual may be improved by increasing the sensory demands placed upon on that individual while the individual performs a physical training task. For example, the quantity and/or quality of visual information available to an individual may be reduced while the individual performs the training task. In such an example, the individual will improve his or her skills in processing the low amount/low quality visual information available, which may involve physiological changes to the ocular system and/or adaptation of the neurological systems involved with vision. Similarly, the task performed under increased sensory demands may result in increased physical strength, improved quickness, greater explosiveness, enhanced agility, etc.

In addition to providing an eyewear device, such as a visor or glasses, that varies the quantity and/or quality of visual information provided to an individual training while wearing the device, systems and methods in accordance with the present invention may provide sensors to measure the effect of the training. The effect of training may be a count of successes (for example, the number of targets hit), but may additionally/alternatively measure the physiological and/or physical response of the individual to the training.

By using sensors to measure the performance and/or physiological response of an individual during training, the sensory training and/or the athletic/rehabilitative training may be adjusted in order to maximize the benefit of the training without discouraging the individual. Eyewear may use one or more lens that controls and varies the quantity and/or quality of visual information available to the individual wearing the eyewear. Eyewear may also provide a display component that provides information and/or instructions to the individual. A control unit may vary the difficulty of training based upon performance and/or physiological measurements by adjusting the quantity and/or quality of visual information provided to the individual and/or by providing instructions to the individual via a display component that increase or decrease the difficulty of the physical training program to be executed by the individual.

A variety of sensors may be used to detect or measure aspects of an individual's performance. For example, a pedometer may simply measure the steps taken by an individual wearing the pedometer. In many applications, data more descriptive of an individual's performance than simply the number of steps taken may be desired. Sensor(s) may be worn by an individual, may be integrated with equipment used by the individual, may be freestanding, and/or may comprise multiple components that may be distributed both on the individual and elsewhere.

One example of a sensor that may be used by an individual for training is a heart rate monitor. A measured heart rate may be used to quantify the exertion of the individual. A target heart rate may be used as an objective for achieving a certain level of cardiovascular training, while for other purposes a maximum desired heart rate may be set to prevent overtraining or for other purposes.

Position monitors, such as global positioning systems (GPS), may be used to determine both the location of the individual at any given instant and to record a distance traveled or route covered by the individual during training. While GPS typically requires that activities occur in an open space permitting the GPS device to receive signals from orbiting satellites, other positioning systems may use beacons or other sources at known locations (fixed or moving) to determine the location of a positioning system unit. Some positioning systems may use multiple cameras to locate an individual during training and/or to track the movement of an individual during training, with a computing device executing instructions retained in a non-transitory medium combining the images from multiple cameras to locate an individual's position during the training.

Accelerometers, inertial sensors, pressure sensors, and/or force sensors may be used to measure the movements, pressures, and/or forces generated by an individual during training and/or the stability or balance of an individual during training. For example, pressure sensors and/or force sensors may be integrated with or inserted into an individual's shoes to measure pressure and/or force produced by an individual, potentially both in terms of magnitude and direction. In some examples, an individual may stand on a platform or other device with pressure and/or force sensors integrated to perform a training exercise. Accelerometers and/or inertial sensors may be integrated into an individual's garments and/or equipment, but additionally/alternatively may be detachably affixed to athletic equipment, a garment, or the individual's body. By combining multiple sensors within a system, the movement of particular portions of an individual's body and parameters describing the individual's focus, stress, and other aspects of performance may be measured and/or detected. For example, pressure sensitive sensors integrated (permanently or temporarily) into an individual's shoes may provide stability data while accelerometers affixed to an individual's arms may provide data describing the swing of a golf club, baseball bat, tennis racquet, or other piece of sports equipment. Accelerometers or other types of sensors may be integrated into equipment as well. For example, a ball, bat, club, racquet, or other item of sports equipment may have sensors permanently or temporarily integrated with the equipment to measure its movement during training.

In some examples the movement of portions of an individual's body during training and/or the movement of sports equipment by an individual during training may be measured without the use of integrated sensors such as accelerometers. Motion capture systems may be used to record the movement of one or more part(s) of an individual's body and/or equipment used by an individual. In some examples, motion capture systems utilize markers affixed to the individual and/or the equipment and one or more camera(s) and an associated computing system executing computer readable code in a non-transitory form to detect those markers in space and track their movement. Other types of motion capture systems may not require any type of marker to be affixed in order to detect and measure motion. For example, some systems use multiple cameras operating in the visible or other portions of the spectrum to capture images and one or more computer processor to identify individual(s) and/or equipment in the captured images and to measure the movement(s) of individual(s) and/or equipment during an athletic competition, a training session of any kind, and/or other situations. By way of further example, some motion capture systems use multiple infrared sensors and/or laser sensors to detect the outline of a person's body and combine multiple infrared images in order to obtain a three dimensional representation of the person's body in space. Any portion of the spectrum other than infrared and visible light as described in such examples herein, may be additionally/alternatively used in a motion capture system. Yet other types of motion capture systems may use beacons affixed to the individual at desired anatomical locations and/or to sports equipment that transmit a signal that is detected and used to determine the location of that beacon at a given time and to detect the movement of that beacon through space over time.

Eye tracking systems may measure the movement of an individual's eyes and/or the focus of the individual's eyes. Eye tracking systems may be integrated into eyewear or headwear worn by the individual during training. Eye tracking systems may be part of a visual training system, but may also be a separate system.

Other types of sensors that may be used to measure aspects of an individual's physiology may be used. Measurements of an individual's physiological response to training may be an indication of the individual's performance, fitness level, cognitive stress, and/or attentional focus. For example, respiration rate, blood pressure, skin temperature, forces or pressures generated, perspiration rate, eyelid blink rate, electrodiagnostics, facial tension, palpebral fissure, or any other medical/biological parameter may be measured.

Performance data describing training and/or competitive success may also be measured using sensors. The relative success of a training exercise itself may be measured. For example, the accuracy of a rifle shot, the speed and/or accuracy of a baseball/softball pitch, the correct read of an American football defense by a practicing quarterback, the accuracy of a golf putt, or the relative success in performing a training task may be measured and detected.

Information may be displayed to an individual using at least one display component provided within the visual training system. A display may comprise a region of one or more of the lenses able to display text, graphics, or other information. A display may be projected onto a lens, but alternatively/additionally a display may be generated on or within the lens itself. A display may be alphanumeric, pictographic, or in any other form that communicates information to an individual. Alternatively or additionally, a display element may be incorporated into a portion of a frame retaining a lens or affixed to a frame and/or lens. Multiple displays may be used in eyewear in accordance with the present invention.

Information displayed may comprise training instructions or directions. For example, an individual may follow a pre-programmed training regimen by following the directions displayed. Such a training regimen may be designed to improve the visual skills of an individual, but may also be used to develop physical skills in conjunction with the training of visual skills. For example, an individual may perform rehabilitative or sport related training activities while a visual training device adjusts the quantity and/or quality of visual information available to the individual. The display may indicate to the individual which training activity to engage in next, the number of repetitions remaining, etc. The display may also be used to instruct the individual to increase or decrease the difficulty of physical training tasks performed, or to change the training task performed.

Information displayed may additionally or alternatively comprise feedback regarding some aspect of an individual's performance during training. For example, the accuracy of a shot, the speed of a thrown ball, and the power of a swing are some types of information that may be displayed to an individual via a display during training. Information displayed may additionally or alternatively describe a physiological, kinematic, or other aspect of an individual's performance. For example, stability data may be displayed for a golfer practicing chipping or other golf shots; heart rate and/or blood pressure information may be displayed to a biathlete practicing transitioning from skiing to shooting; eye tracking data may be displayed for a quarterback practicing reading defenses; any of a variety of other types of data or other information may be displayed to a training individual. Information displayed may be raw data, such as numbers represented measured heart rate or blood pressure, but may also be processed in some way in order to be readily understood by a training individual. For example, balance or stability data may be indicated using a depiction of an individual's feet and a dot illustrating the individual's center of gravity. Physiological and/or performance data may be combined into a score or other indicator descriptive of an individual's training progress.

The quantity of visual information available to an individual may be varied using a lens switchable between a substantially transparent state and a substantially opaque state. All or part(s) of the lens may be switchable, and optionally individual elements or portions of a lens may be addressable to be switched between an opaque and a transparent state. The relative times for which a lens is in a transparent state versus an opaque state may be a measure of the quantity of visual information received by the individual. Additionally/alternatively, the relative amount of an individual's visual field occupied by a portion of a lens in a transparent state versus the amount of an individual's visual field occupied by a portion of a lens in an opaque state may be a measure of the quantity of visual information received by the individual. In some examples, a lens may be provided for each eye of an individual, with each lens being controlled distinct from the other lens.

The quality of visual information available to an individual may vary be varied by adjusting the optical power of the lens, by altering the microstructure of the lens to blur light passing through it, by only partially reducing the transparency of the lens, or through any other means that reduces the contrast, crispness, and/or clarity of visual information perceivable through the lens. Individual regions or portions of a lens may be individually addressable to vary the quality of the visual information transmitted by a lens.

One or more lens may be mounted to be worn over one or more eye of an individual for training. A visor or shield design eyewear may provide a single lens, while a glasses frame may provide two lenses, one lens per eye. A lens may optionally provide visual correction for an individual, and may have optical properties to avoid distortion of an image to an individual wearing the lens(es). A lens may optionally provide impact protection, protection from ultraviolet light, operate as sunglasses, filter some or all wavelengths of light to improve (or to impair, for training purposes) a wearer's perception of particular visual cues, etc.

An eyewear controller may control and/or power the one or more lens as appropriate to adjust the quality and/or quantity of visual information available to an individual. The eyewear controller may also control the display of information in a display component viewable by an individual during training. The eyewear controller may be integral to the glasses, visor, or other structure retaining the lens(es) in position during training. Similarly, a battery or other power source may be provided to power changes in quantity and/or quality of visual information available through lens(es). At least one communication interface may be provided as well, in order to permit the eyewear controller to interact with a control unit, sensors that measure performance or physiological parameters during training, and/or other devices.

By limiting the quantity of visual information available to an individual during training, an individual may develop his or her visual and related abilities to perform with that reduced level of information, thereby increasing the individual's performance during competition when a full amount of visual information is available. Similarly, by reducing the quality of the visual information available to an individual, the individual's visual and related abilities may increase to compensate for the lower quality information available during training, thereby improving athletic performance during competition when the quality of visual information available to the individual has not been intentionally impaired. The time during which the quantity and/or quality of visual information is limited may be varied as well, determined for example to reduce quality and/or quantity of visual information available during different times of a training task, for example based upon sensor measurements, to more particularly develop an individual's abilities for specific aspects of a training task. Further, limiting visual information available to an individual, either in quality or in quantity, may assist the individual in better integrating other senses, such as auditory and/or proprioceptive senses, into her or his athletic performance.

The present invention may utilize measurements from both wearable sensors and other sensors. For example, optical, infrared and/or other types of markerless position measurement systems may be used to measure performance of an individual undergoing training/testing in accordance with the present invention. The present invention may use any type of system that provides further measurements regarding the physical location of a user and/or portions of a user's anatomy, however, whether markerless or not. For example, systems for measuring and tracking position using infrared signals, magnetic measurements, measurements using visible light, or other means may be utilized in accordance with the present invention. Such measurements from non-wearable sensors and systems may be incorporated in real-time with measurements made by wearable sensors, but may also be used subsequent to a testing/training program as part of a record of an individual's performance in conjunction with measurements made by the wearable sensors and/or the testing/training program dynamically implemented by a control unit.

In order to fuse data collected using wearable sensors and data collected from other sensor systems to dynamically adjust a testing/training program in real-time, the time lag involved with communicating a sensor measurement to a control unit may be carefully measured and controlled for in selecting and timing the presentation of symbols providing instructions and/or in varying the quantity/quality of visual information provided to an individual as part of a program. Clock data may be used to provide time stamps for measurements made by different types of sensors. In some examples, time stamps may be used for all measurements received by a control unit in order to place those measurements in an appropriate sequence with measurements made by other sensors. In other examples, a calibration cycle may be performed periodically to determine the relative time lag encountered for different types of sensors, with appropriate adjustments made by the control unit to account for the anticipated time lag for the individual types of sensors (or even each individual sensor) used in a particular implementation of the present invention.

The present invention may further use a combination of multiple types of sensors, multiple types of symbols and actions directed by those symbols, and/or multiple ways of varying the physical and/or sensory difficulty of a program. For example, the present invention may utilize both inertial sensors worn by a user and force sensors integrated into a user's shoes in conjunction with infrared location sensors external to the user that communicate with a processing unit to vary both the physical difficulty and the sensory difficulty of a testing/training program in accordance with the present invention.

A control unit may communicate with wearable sensors, and optionally other sensors or measuring systems, to receive measurements indicating the performance of an individual during a testing/training program in order to dynamically adjust the difficulty of the training program based upon the real-time performance of the individual. The difficulty of the training program may be adjusted by varying the physical difficulty of the program activities, by adjusting the sensory difficulty of the activities through altering the quantity and/or quality of visual information provided, or a combination of some or all of these.

A control unit may be integrated into eyewear in accordance with the present invention, but may alternatively be worn on another portion of an individual participating in a training program or located at a convenient location in the area used for training. In some examples, a control unit may be integrated into a wearable sensor. However, a control unit may be distinct from eyewear, sensors, and other aspects of systems in accordance with the present invention. A control unit may exchange information with one or more heads-up display, lens(es) that adjusts the quantity and/or quality of visual information transmitted, and wearable sensors via any appropriate communication media and/or protocol. For example, if a processing unit is physically incorporated into eyewear that provides the one or more heads-up display and lens(es) that may be adjusted to provide varying amounts quantity and/or quality of visual information to a user, then the processing unit may be electrically coupled to the one or more heads-up display and be one or more lens. In the same example, inertial sensors optionally incorporated into the eyewear to provide measurements of an individual's head movement may further be electrically coupled to the control unit, but sensors worn elsewhere by the individual may communicate wirelessly with the control unit.

Sensors, eyewear with one or more lens controlling the quantity and/or quality of visual information available to the individual, and any control unit managing, recording, and/or adjusting training may communicate over various mediums and using any protocol. For example, a sensor may communicate wirelessly (via Bluetooth, an 802.11x protocol, or other standard) with a control unit. However, wired connections may be used in accordance with the present invention. A control unit may communicate wirelessly with an eyewear controller and/or sensors that measure performance and/or physiological parameters of an individual during training. A control unit and an eyewear controller may be discrete units, for example with the eyewear controller integral to the eyewear retaining one or more lens and the control unit operating on a special purpose or general purpose computing device. Alternatively, a control unit and an eyewear controller may comprise a single unit. While a division of functionality between an eyewear controller and a control unit are described in examples herein, in various implementations the functions performed by an eyewear controller(s) and a control unit(s) may be different than described herein, and may be distributed to additional or different devices.

The training conditions experienced by an individual may be varied based upon the relative success and/or physiological response of an individual during training. Sensors may measure the performance of an individual and/or the physiological condition of an individual, and appropriate adjustments to the training program may be made to increase the difficulty of training, decrease the difficulty of training, and/or change the nature of training. The training program may be adjusted using a display component to provide instructions to an individual to alter the training program. The alteration of the training program may be to increase the difficulty of training to maximize positive training effects, decrease the difficulty of training to avoid discouragement, and/or to change the nature of training to address a different ability or skill. For example, an individual may be instructed to move to a different drill, to use a different target for throwing/shooting/kicking/putting/driving/etc., or to otherwise alter the training regimen. The visual aspects of the training may also be adjusted based upon measured performance and/or physiological data. The quantity of visual information may be increased or decreased. The quality of visual information may additionally/alternatively be increased or decreased. For example, if an individual has mastered a training exercise with first level of visual information providing a given quantity and/or quality of visual information, the control unit may adjust the training to a second level of visual information providing a decreased quantity of visual information or a lower quality of visual information. On the other hand, if an individual is struggling with a given level of visual information, the quantity and/or quality of visual information may be increased. In some examples, the quality of visual information may be decreased while the quantity of visual information may be increased, or vice versa, in order to train different aspects of an individual's visual or related athletic abilities. The quantity of visual information may be adjusted by decreasing the amount of time during which a lens is in an entirely or partially transparent state, by decreasing the area of a lens that is in a transparent state, and/or (if a lens is provided for each of an individual's eyes) opening only a single lens into a transparent state at a time.

An individual may be directed to and engage in movements associated with a program through the display of symbols on one or more display, such as a heads-up display. Instead of and/or in addition to one or more heads-up display, a discrete display (such as a monitor, an image projected onto a screen, etc.) may be used to display symbols in accordance with the present invention. Symbols used to communicate actions of a training program to an individual may be simple or complex. For example, arrows may be used as symbols to indicate a direction in which an individual should step, turn, jump, were otherwise move. By way of further example, symbols may comprise letters, words, arithmetic problems, depiction of items, and/or any other method of communicating visually with an individual to describe the next action to perform as part of a testing/training program. By way of yet further example, the instructions provided may additionally/alternatively provide directions relating to time instead of or in addition to directions relating to space. For example, an arrow or other indicator may instruct an individual to speed up or slow down a movement, such as a running pace, repetitive motion, breathing pattern, etc.

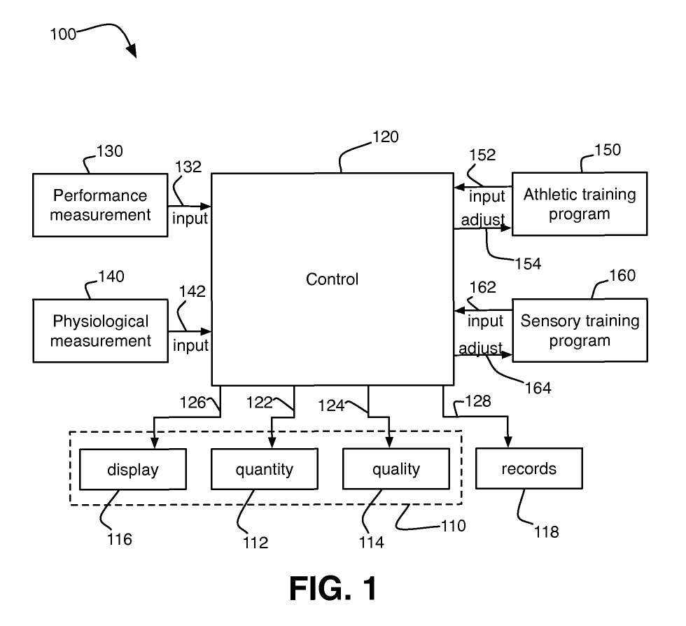

FIG. 1 illustrates an example of a system 100 in accordance with the present invention. An eyewear component 110 may control the quantity 112 of visual information provided to an individual and/or the quality 114 of visual information provided to an individual. Eyewear component 110 may also provide a display 116 to provide visual information to an individual. Display 116 may provide information to an individual describing the performance of the individual during training, the physiological measurements of the individual during training, information describing the quantity or quality of sensory information provided to the individual during training, information describing the difficulty of the physical training, or other information (such as time remaining in training, receptions of a drill remaining, a summary of physiological or performance metrics, a description of the quantity/quality of visual information being provided by the eyewear to the individual, etc.). Display 116 may additionally/alternatively provide directions, instructions, or other information to an individual. Performance measurements 130 and physiological measurements 140 may be made by one or more sensors.

A control unit 120 may receive performance measurement 130 inputs 132 and/or physiological measurement 140 inputs 142. A control unit 120 may also control via signal 122 the quantity 112 of visual information available to an individual, may control via signal 124 the quality 114 of visual information available to an individual, and may control via signal 126 the information displayed 116 to an individual. A control unit 120 may control the operation of eyewear components 110 directly or via an eyewear controller.

A control unit 120 may receive an input 152 of a physical training program 150 to be performed by an individual. A physical training program may define or describe, for example, the drills, tasks, exercises, or other training actions to be undertaken by an individual. Based upon criteria, such as performance measurements 130 and/or physiological measurements 140, a control unit 120 may adjust 154 a physical training program 150.

A control unit 120 may additionally/alternatively receive an input 162 of a sensory training program 160. A sensory training program may define or describe, for example, the quantity 112 and/or quality 114 of visual information an individual will receive through an eyewear component 110 during training. A sensory training program 160 may be coordinated with a physical training program 150, but such coordination is not necessary. Based upon criteria, such as performance measurements 130 and/or physiological measurements 140, a control unit 120 may adjust 164 a sensory training program 160.

One or more record 118 may be made of the physical and/or sensory training of an individual. A record 118 may describe one or more of the individual engaging in a training program, the time or date of the training, the physical training program 150 executed, the sensory training program 160 executed, performance measurements 130 made during training, and/or physiological measurements 140 made. A record 118 may be maintained in an appropriate computer readable form in any type of memory or storage device. A record 118 may be maintained within a control unit 120, within an eyewear component, or at another location. One or more records 118 may be periodically copied or moved to a database or other storage system.

While control unit 120 is shown in the example of FIG. 1 as separate from eyewear component 110, control unit may be integral with eyewear component 110. Further, control unit 110 may comprise one or more computing devices having a processor executing computer readable instructions from one or more non-transitory media to operate as described herein.

Adjustments of a training program may relate to the physical training tasks performed and/or the quantity of visual information 112 and/or the quality of visual information 114 available to an individual. For example, if performance measurements 130 and/or physiological measurements 140 indicate that an individual has been successful at a task of a particular level of difficulty, the difficulty of a subsequent training task may be increased in one or more fashion. On the other hand, if performance measurements 130 and/or physiological measurements 140 indicate that an individual has not been successful at a task of a particular level of difficulty, the difficulty of a subsequent training task may be decreased.

For example, a sensor may determine that a basketball player shooting a ball from a particular location on the floor with a particular quantity and quality of visual information has reached a threshold level of success, such as, for instance, hitting five consecutive shots. In such an example, the basketball player may be instructed to move further from the basket, the quality of the visual information provided to the basketball player may be decreased, and/or the quantity of visual information provided to the basketball player may be decreased. Conversely, a lack of success (such as a basketball player missing a given number of shots) may result in the training becoming easier by instructing the individual to move closer to the basket, increasing the quality of visual information available to the individual, and/or increasing the quantity of visual information available to the individual. Of course, the present invention is not limited to any particular sport or training task, but may be applied for any type of sport, rehabilitation, and/or other training, and may involve any type of physical training task associated with a sport or type of rehabilitation.

In some examples, some portions of a training program may not be adjusted based upon physiological or performance measurements. For example, if sensors indicate that an individual is struggling to maintain his or her balance, the sensory challenge and/or the physical challenge may be decreased, while the sensory and/or physical challenge may be increased if sensor measurements indicate that the individual has successfully maintained his or her center of balance within a desired degree of stability.

In some instances an assessment may be obtained for an individual to permit the individual to evaluate his or her improvement relative to a prior assessment or in comparison to other individuals. In some examples, such an assessment may be used to establish a baseline for subsequent training by that individual. Adjustments to training difficulty, whether to increase or to decrease the difficulty of training, may be made dynamically during training but may additionally/alternatively be made between training sessions and/or during breaks of a training session. In some examples, certain types of adjustments to training difficulty may be made dynamically during training, such as changes in the quality and/or quantity of visual information available to an individual, while other types of adjustments to training difficulty, such as the parameters of a training task, may be adjusted during breaks in training.

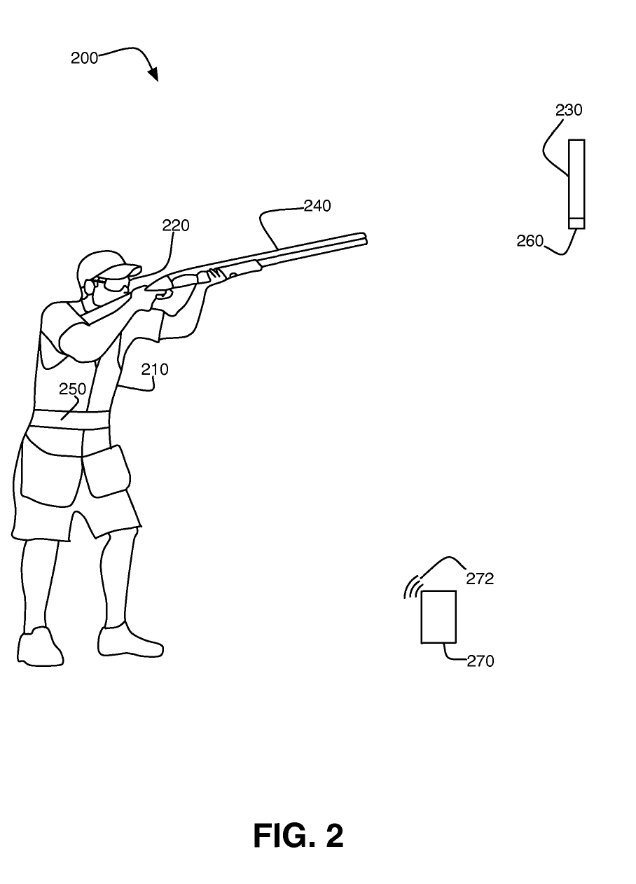

FIG. 2 illustrates an example individual 210 training using a gun 240 to shoot a target 230 using a system 200 in accordance with the present invention. An eyewear component comprising glasses 220 control the quantity and/or quality of visual information available to individual 210. A sensor 260 associated with target 230 may be used to provide a performance measurement by measuring the accuracy of ball 240 in striking target 230. Sensor 260 may be physically affixed to target 230, as illustrated in the example of FIG. 2, and may detect a vibration, electrical signal, or any other measurement indicative target 230 being hit, but additionally/alternatively sensor 260 may be physically disconnected from target 230 and may utilize sound detection or other means to determine whether target 230 has been successfully hit. A sensor 250 associated with individual 210 may provide one or more physiological measurement by measuring the heart rate, blood pressure, movement, stability, or other data describing biological or medical condition of individual 210. A control unit 270 (illustrated as a discrete component for illustrative purposes in the example of FIG. 2) may communicate wirelessly 272 with glasses 220, performance sensor 260, and/or physiological sensor 250. Based upon performance measurements and/or physiological measurements, control unit 270 may adjust the quantity and/or quality of visual information received by individual 210 through glasses 220. Optionally, control unit 270 may use a display component within glasses 220 to display information or instructions to individual 210. Instructions provided to individual 210 may increase or decrease the difficulty of physical training tasks in response to performance measurements and/or physiological measurements.

While described in terms of athletic training, the present invention may be used for any type of physical activity, such as but not limited to rehabilitation to improve, restore and/or maintain physical and/or sensory skills that have been or are impaired by injury, illness, and/or age. Such rehabilitation need not be sport related. Further, systems and methods in accordance with the present invention may be helpful in assessing the degree and type of impairment experienced by an individual.

The example of the present invention illustrated in FIG. 2 is not limited to any particular sport or type of training, and may be used for skills, such as basic balance and coordination, that are needed for rehabilitation services. The performance and/or physiological data measured may vary from the examples described herein. In some examples, systems and methods in accordance with the present invention may implement only some types of sensors, such as only performance sensors or only physiological sensors or only certain types of performance or physiological sensors. Similarly, some implementations of the present invention may adjust only the quantity or only the quality of visual information, or may only restrict one of the quality or the quantity of visual information provided.

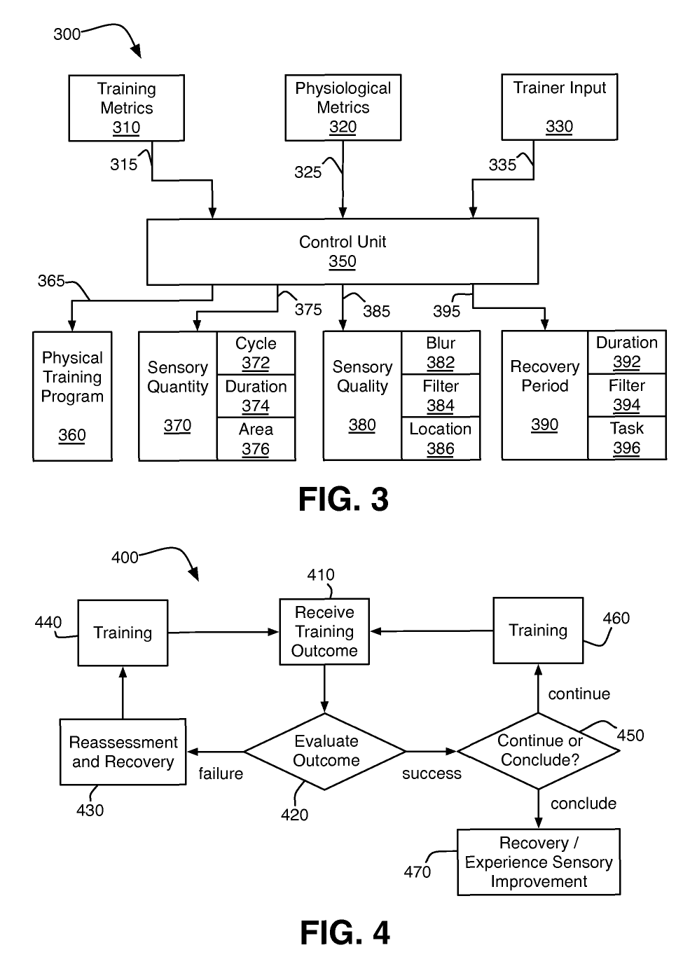

Referring now to FIG. 3, a further example of a system 300 in accordance with the present invention is illustrated. Training metrics 310 may be performance metrics determined, for example based upon sensor measurements, and communicated to a control unit 350 via a connection 315. Training metrics 310 may comprise any type of measurement of the relative success of a training task, such as hitting a shot, making an accurate throw, or a coach or other trainer affirming that a task was successfully completed (for example, using a device such as a mobile phone, computer, remote control, or other device to indicate the successful or unsuccessful completion of a training task). Training metrics may be binary, indicating either “successful” or “not successful” in some way, but may also be relative. For example, a training task may be repeated for a certain number of repetitions, such as five, with success indicated by the number of successful repetitions. Additionally/alternatively, a training metric may comprise a metric such as proximity to a target, either in an absolute sense (for example, six centimeters from the target) or in a relative sense (for example, the second ring of the bulls eye). Further, a training metric may comprise a time of completion, a force generated, a degree of rotation of the individual's body or a piece of equipment, a distance covered, or any other description of the performance of an individual engaged in a training task. More than one metric may be collected as part of training metrics 310.

Still referring to FIG. 3, physiological metrics 320 may be collected and communicated to control unit 350 via connection 325. Some examples of physiological metrics 320 are described herein, but any measurement describing the physiological response of an individual to training may be used in accordance with the present invention. Further, more than one physiological metric 320 may be collected in accordance with the present invention.

Trainer input 330 may optionally be communicated to control unit 350 via connection 335. Trainer input 330 may comprise evaluations by a trained individual (such as a coach, doctor, or physical therapist) of the performance of an individual training in accordance with the present invention, but need not comprise training metrics 310. In some examples, trainer input 330 may comprise an input from the individual training that assesses how the individual subjectively feels about the training process. Trainer input 330 may comprise inputs for application in subsequent training sessions, for example. In some examples, a trainer input 330 may immediately interrupt a training session, for example to immediately remedy a training error, such as may occur if the individual training is performing a training task incorrectly, or to protect the health, safety, or wellbeing of the individual training.

One or more of the training metrics 310, physiological metrics 320, and trainer input 330 may be omitted in accordance with the present invention. For example, if a particular implementation of the present invention is more concerned with physiological evaluation and/or training, both the training metrics 310 and/or trainer input 330 may be omitted. On the other hand, if a particular implementation of the present invention is primarily focused on improving training outcomes through improved sensory skills, physiological metrics 320 and/or trainer input 330 may be omitted. In yet other examples, only trainer inputs 330 may be used.

The control unit 350 may control various aspects of physical and/or sensory training based upon prior programming and/or received data such as the training metrics 310, physiological metrics 320, and/or trainer input 330 received. The physical training program 360, which may be communicated to an individual using a display component, an auditory signal, or through other communication means, may be varied to best serve the training objectives in light of the received data. Similarly, the sensory quantity 370 and/or sensory quality 380 available to an individual may be adjusted in light of the received data to provide optimized training. Additionally/alternatively, the recovery period 390 may be adjusted based upon the received data.

Sensory quantity 370 may be adjusted in various ways. For example, the cycle 372 in which the amount of sensory information available to an individual is restricted may be adjusted. The cycle 372 may comprise a frequency, for example the frequency at which all or part of the lens(es) obscure an individual's vision. Sensory quantity 370 may also be adjusted by changing the duration 374 for which sensory information is, or is not, provided to an individual. For example, within a given cycle 372 lens(es) may transmit visual information to an individual for only a certain period of time or a percentage of the cycle. A longer duration 374 without visual information may be more stressful to an individual than a shorter duration 374 without visual information. Further, the area 376 in which lens(es) limit visual information may be varied. For example, lens(es) may limit an individual's entire field of view, but alternatively may limit only a fractional portion or percentage of an individual's field of view. While the portion of a field of view limited may alter the stress applied to an individual in training, particularly if the portion is contiguous rather than distributed over the entire field of view in a checkerboard fashion, generally the greater the area without sensory information provided the greater the sensory stress placed upon an individual. Another example of limiting the quantity of visual information provided to an individual is to limit visual information available to a single eye at a time.

Sensory quality 380 may also be adjusted in various ways. For example, a visual signal may be degraded using a blur 382 that de-focuses light passing through the lens(es). A blur 382 may be controlled by adjusting the curvature, power, and/or distribution of particles within lens(es). By way of further example, a filter 384 that selectively removes light passing through lens(es) based upon the wavelength of that light may make the visual information provided to an individual either higher quality or lower quality, depending upon whether the wavelengths removed by filtering are extraneous noise or critical information to the task being performed.

A recovery period 390 may be provided during which no or little reduction in either the quantity 370 and/or quality 380 of visual information is performed. A recovery period 390 may be useful to facilitate desensitization to the physical and/or sensory stress associated with training, or even to avoid negative physiological responses, such as nausea and dizziness, that may occur in individuals engaging in perceptual stress training. Based upon the received information, the control unit 350 may adjust the duration 392 of a recovery period. Further, a filter 394 applied for a recovery period 390 may vary based upon the received information, as some filters may be particularly soothing or beneficial to an individual in some circumstances. Additionally/alternatively, the task(s) 396 performed during a recovery period 390 may vary based upon the received information.

Variations of a recovery period 390 in accordance with the present invention may differ based upon the purpose of a particular recovery period 390. For example, if a recovery period 390 is intended to permit an individual to recover from negative physiological metrics 320, the duration 392 may be extended until sufficiently improved physiological metrics 320 and/or a trainer input 330 indicating a readiness to continue is received by control unit 350. If a recovery period 390 is intended to correct a training error indicated from a training metric 310 and/or a trainer input 330, may be relatively short, or may last until a training input 330 indicating a readiness to resume training is received by control unit 350. In some examples, a trainer input 330 may comprise an input from the individual training or another person supervising the training to indicate that he or she is ready to resume training and/or that the individual is not ready to resume training. By way of further example, if a recovery period 390 is intended to enhance the confidence of an individual training and/or to provide an immediate improvement to the performance of the individual, an appropriate task 396 may be performed in order for the individual to experience the positive effects of the sensory training. A recovery period 390 may be abrupt or gradual. For example, an individual may gradually receive increasing quantities of visual information during the beginning or the entirety of a recovery period 390. For example, an individual working to improve balance skills may develop balance abilities through training with peripheral visual information reduced or entirely eliminated, and during a recovery period 390 some or all of the peripheral visual information may be restored to the individual.

Referring now to FIG. 4, an exemplary method 400 in accordance with the present invention is illustrated. Method 400 may receive a training outcome in step 410. A training outcome may comprise, for example, one or more training metric, one or more physiological metric, and/or one or more trainer input. The training outcome may be evaluated in step 420. Step 420 may involve comparing the training outcome to predefined parameters or goals, to an individual's prior performance, a binary determination of success, or any other determination. If the outcome of evaluation 420 is that the training task was a failure, method 400 may proceed to step 430 of reassessment and recovery in order to allow the individual to improve upon his or her performance. Method 400 may proceed from step 430 to a training step 440. The training of step 440 may be at a different degree of difficulty, such as lower difficulty, than training previously performed unsuccessfully. If the result of evaluation step 420 is that the training task was a success, method 400 may proceed to step 450 to determine whether to continue or conclude that component of training. Step 450 may determine to conclude a component of training if, for example, an individual has successfully completed a training task based upon a predetermined success threshold. A success threshold may be related to attaining a particular training metric, such as successfully completing five consecutive tasks. A training metric may comprise any measured physiological or performance metric, such as stability data, and a corresponding success threshold may be based upon that data. For example, stability data may be collected while all or part of an individual's peripheral visual information is restricted, and the stability data may be analyzed to provide an assessment of the individual's balance relative to a success threshold. If the determination of step 450 is to continue with training, method 400 may proceed to an additional training step 460. The training of step 460 may be more or less difficult than previous training, for example by increasing difficulty after training is performed successfully and/or decreasing difficulty after training is performed unsuccessfully. For example, if an individual is training to improve stability and balance, the amount of peripheral visual information provided may be decreased after a success and increased after a failure, with such changes in the available visual information being either gradual or sudden. After a training step, such as training step 460 and/or training step 440, method 400 may return to step 410 to receive training outcomes. If step 450 determines to conclude the component of training, method 400 may proceed to step 440 of providing a recovery period during which the individual may experience a sensory improvement from the training. In some examples, measurements of the individual's performance may be made during a recovery period to provide an indication of the efficacy of the training. Method 400 may thereafter conclude or resume with a training step, potentially training addressing a different skill.

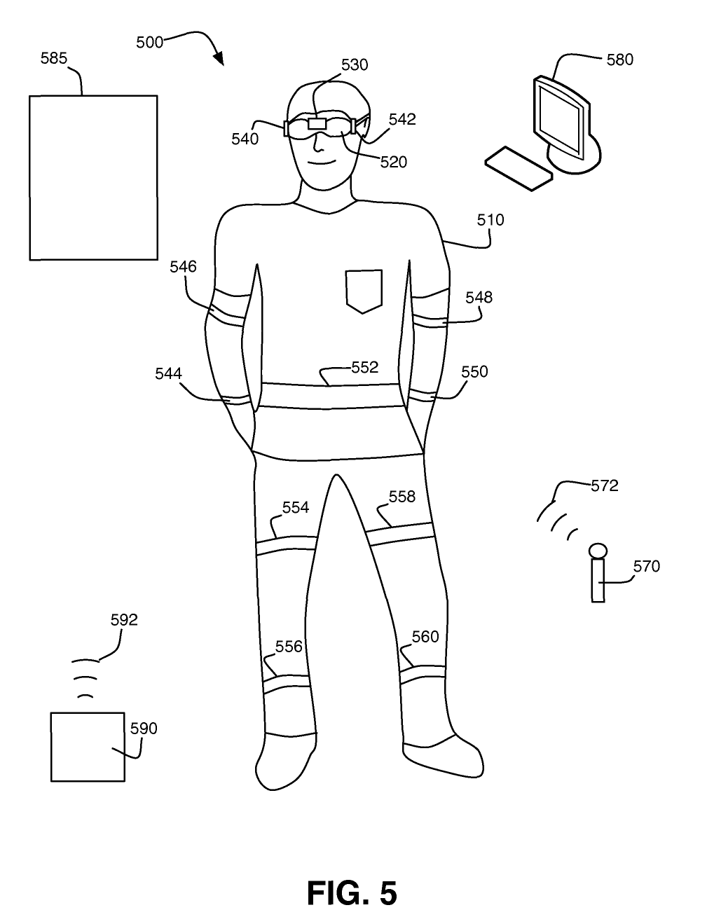

FIG. 5 illustrates a system 500 in accordance with the present invention for administering a program to train the physical, neurological, sensory, and/or other abilities of an individual 510. Individual 510 is wearing eyewear 520 with an integrated control unit 530. A first sensor 540 and a second sensor 542 are integrated into eyewear 520.

Additional sensors are integrated into wearable technology worn by individual 510. In the example illustrated in system 500 of FIG. 5, a first wrist sensor 544, a first elbow sensor 546, a second elbow sensor 548, a second wrist sensor 550, a waist or torso sensor 552, a first knee sensor 554, a second knee sensor 558, a first ankle sensor 556, and a second ankle sensor 560 are illustrated. However, more, fewer, and/or different sensors than those depicted in FIG. 5 may be used in accordance with the present invention. The plurality of sensors illustrated in FIG. 5 may be in communication with control unit 530 via any wired or wireless communication protocol. The sensors may all be of the same type, but may be of different types. For example, eye tracking sensors, inertial sensors, pressure sensors, and perspiration sensors may all be used, as may any other combination of wearable sensors.

Still referring to FIG. 5, at least one external measurement system 570 may optionally be provided to record further data regarding the performance of individual 510. Measurement system 570 may use signals 572 to make measurements describing the performance of individual 510 and portions of the anatomy of individual 510 during a testing/training program. Signals 572 may be, for example, infrared, visible light, radio frequencies, etc. Further, signals 572 may comprise light or other wavelengths of electromagnetic radiation reflected off of markers worn by individual 510. Further, signals 572 may comprise sound waves, ultrasonic waves, subsonic waves, were any other type of signal.

Further, system 500 may provide external stimuli 592 created by a generator 590. One example of a generator 590 is a metronome that provides a rhythmic stimuli 592 for individual 510 to comply with in performing a physical activity, but any other type of stimuli 592, predictable or unpredictable, may be used in conjunction with the present invention to provide a varying difficulty of a testing/training program. A stimuli 592 may comprise a distraction to individual 510, but may additionally provide a second input directing individual 510 in the actions of a testing/training program.

Still referring to FIG. 5, one or more external computing device 580 may be used in real-time or non-real-time coordination with a control unit 530, measurement system 570, and/or external stimuli 592 generator 590. In some examples, additional computer 580 may be used to program processing unit 530 and/or to store performance records made by sensors and communicated to processing unit 530 during a testing/training program.

One or more heads-up display may be integrated into eyewear 520 in order to provide program instructions to individual 510. Additionally/alternatively, an external display 585 may be provided to provide program instructions to individual 510 undergoing testing/training in accordance with the present invention.

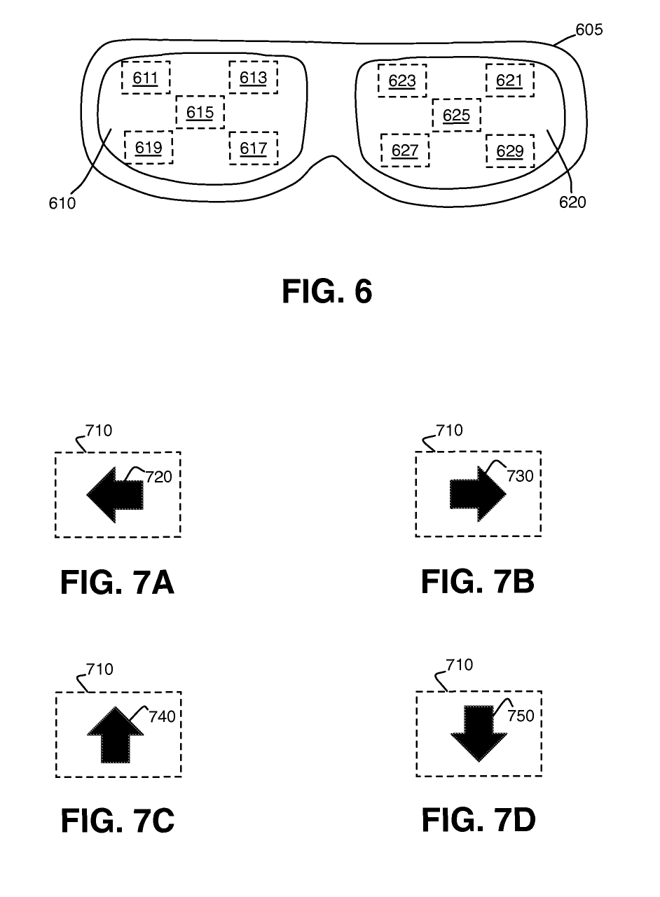

Referring now to FIG. 6, one example of eyewear 620 in accordance with the present invention is illustrated. The example eyewear 620 shown in the example of FIG. 6 provide two lens retained within a frame 605 to be worn as glasses, but a single lens visor or other types of eyewear may be utilized in accordance with the present invention.

In the example of FIG. 6, a first lens 610 and a second lens 620 are retained within frame 605. While not shown in the example of FIG. 6, a control may be incorporated within a frame 605 of eyewear 620 or elsewhere in eyewear 620. As described above, first lens 610 and/or second lens 620 may be controlled by control unit to vary the quantity and/or quality of visual information provided to an individual wearing eyewear 620. Further, eyewear 620 may incorporate one or more heads-up display. The present invention may utilize a single heads-up display, multiple heads-up displays, heads-up displays in amounts and/or arrangements other than depicted in the example of FIG. 6, and/or may use an external display for some or all displaying of symbols to provide instructions to an individual participating in a testing/training program in accordance with the present invention.

For example, eyewear 620 may provide multiple heads-up displays, and the heads-up display used for purposes of providing symbols to instruct an individual in the performance of a training program may be dynamically altered to vary the difficulty of a training program. The change of the heads-up display used to provide a symbol to individual and may be one means of varying the difficulty of a testing/training program in accordance with the present invention, as the degree of unpredictability and a heads-up display used and the location of a heads-up display relative to a typical gaze of an individual may impact the difficulty encountered in performing the actions communicated via symbols displayed on a heads-up display.

In the example of FIG. 6, a first lens 610 provides a first heads-up display 615 in the center of lens 600 and, a second heads-up display 611 in the upper left corner of lens 610, a third heads-up display in the 613 in the upper right corner of lens 610, a fourth heads-up display 619 in the lower left corner of lens 610 and, and a fifth heads-up display 617 in the lower right corner of lens 610. Similarly, second lens 620 may provide a first heads-up display 625 in the center of lens 620, a second heads-up display 621 in the upper right corner of lens 620, a third heads-up display 623 in the upper left corner of lens 620, a fourth heads-up display 629 in the lower right corner of lands 620, and a fifth heads-up display 627 in the lower left corner of lens 620. While first lens 610 and second lens 620 are depicted in the present example as possessing five discreet heads-up displays each, the present invention may utilize a single heads-up display and a single lens, a single heads-up display in each lens, and/or numbers or locations of heads-up displays other than those illustrated in the present example. Other variations of the use of a heads-up display without departing from the scope of the present invention.

In some examples of the present invention, more than one display may be used to convey information to an individual. For example, a first display may display data to the individual, and the displayed data may or may not be descriptive of the testing/training program being performed. Information displayed may be obtained, in whole or in part, using sensors of the system. Examples of information descriptive of the program being performed are heart rate information, success rate for the program thus far, time or number of repetitions remaining for the program or the current portion of the program, etc. In some examples, information may be displayed to increase the sensory and/or neural processing load experienced by the individual, for example to “distract” an individual. Examples of distracting information may be simple lights, irrelevant messages, pictures, text, etc.

Further, more than a single display may be used to provide an instruction to an individual. For example, a first display may be used to instruct an individual to take a first type of action (such as to turn), while a second display may be used to instruct an individual to take a second type of action (such as to crouch or jump), with the differentiation between those displays to identify the correct action to take in response to provided symbols serving as part of the testing/training program.

By way of further example, in some instances a first display may be used to direct an individual as to which of the other displays should be used to receive the next instruction. For example, an arrow or other symbol in a central display may be used to indicate which of a plurality of additional displays will provide the next actionable instruction. The indication as to which additional display should be used to provide the next actionable instruction need not be an arrow, but may use an alphanumeric, pictographic, color, or other designation to indicate which display will provide the next actionable instruction. Additional neural processing by the individual, such as performing a mathematical calculation to attain a number corresponding in some way to the display to be used for the next actionable instruction, may be required in accordance with the present invention in order to increase the neurological processing load for a testing/training program in accordance with the present invention. In such an example, some or all of the non-indicated displays may provide instructions contradicting the instructions given by the indicated display.

As a yet further example, a first display may provide an output that instructs an individual as to whether to follow the instructions given by a different display. For example, a green indicator in a first display may indicate that the individual should follow instructions provided by a second display, while a red indicator in the first display may indicate that the individual should not follow instructions provided by a second display. In some examples, the determination as to whether to follow instructions may be quite taxing, for example determining whether a number displayed or the solution to a displayed mathematical calculation is odd or even.

Referring now to FIGS. 7A-7D, examples of symbols used to communicate actions to perform as part of a training/testing program are illustrated. The present example symbols are illustrative only, and numerous other types of symbols may be utilized. In the example of FIG. 7A, a left arrow 720 may be used, for example, to indicate to an individual to turn, step, jump, or otherwise move to the left. As shown in FIG. 7B, right arrow 730 may be used to indicate such a motion to the right. Up arrow 740 depicted in FIG. 7C may be used to communicate to an individual to move forwards, to jump up, etc., while a down arrow 750 in FIG. 7D may be used to communicate with an individual move backwards, to crouch, or to otherwise engage in a physical act as part of a training program.

Various other types of symbols of more or even less complexity than those depicted in FIGS. 7A-7D may be utilized in accordance with the present invention. For example, heads-up display 710 may provide a representation of an object to be found in an individual's environment, words describing an action to be taken, a color corresponding to a given action, a mathematical problem to be solved with the action to be taken dependent upon the solution to the problem, or any other type of symbolic representation to communicate an action to be taken as part of a testing/training program. Further, the action dictated by a displayed symbol may be unrelated to, or even contradictory to, the symbol displayed, which may be particularly useful to increase the difficulty of a training/testing program. For example, the difficulty of a training program may be increased by instructing an individual to turn in a direction opposite from the arrow displayed on a heads-up display 710.

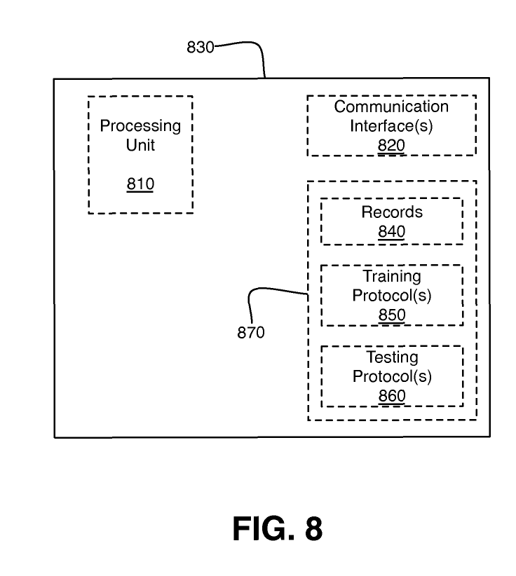

Referring now to FIG. 8, a control unit 830 such as may be used in accordance with the present invention and optionally integrated into eyewear is illustrated. The control unit 830 may provide a computer processor 810 to execute machine readable code retained in a non-transitory storage media to execute a series of steps to administer a dynamically adjustable testing/training program as described herein. The processor may dynamically alter the physical and/or sensory difficulty of a program via the symbols provided on one or more heads-up display and/or the quantity/quality of visual information transmitted via adjustable lens(es). A communication interface 820 may enable control unit 830 and processor 810 to communicate with various sensors, lens(es), heads-up display(s), external computers, external measurement systems, and/or other devices or outputs. A memory and storage component 870 may retain records 840 of sensor measurements and/or programs applied via a heads-up display and/or lenses, computer readable code embodying dynamic training protocol programming 850 to be followed during a training program, and/or computer readable code embodying dynamic testing protocol programming 860 to be followed during a testing program.

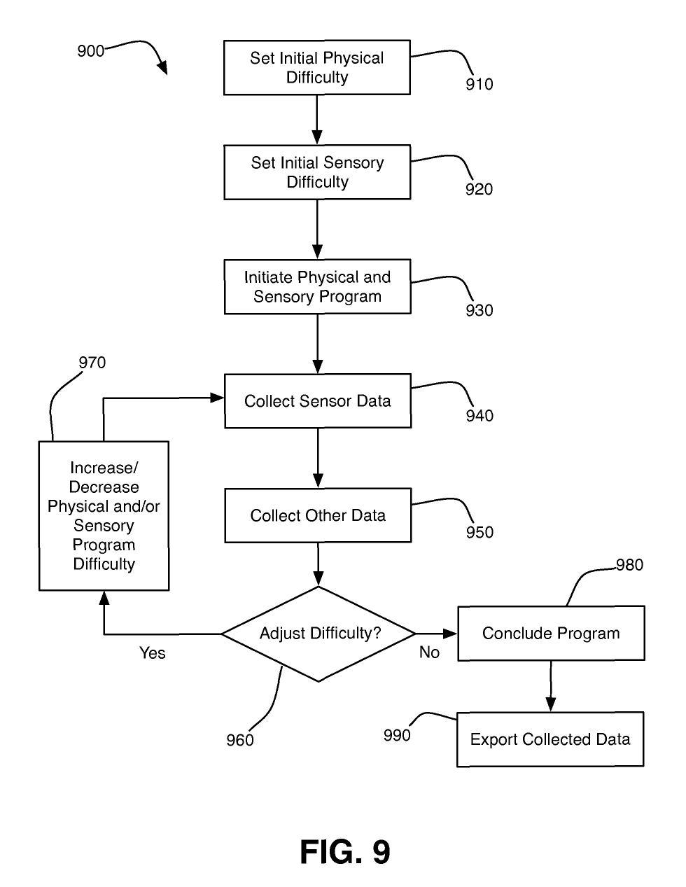

Referring now to FIG. 9, a method 900 in accordance with the present invention is illustrated. Method 900 may begin at step 910 of setting the physical difficulty of a training program. Method 900 may also comprise step 920 of setting the initial sensory difficulty of a testing/training program. The physical difficulty setting step 910 may relate to the physical challenge of the tasks to be performed at the direction of symbols provided on one or more heads-up display, while the sensory difficulty set in step 920 may relate to the quantity and/or quality of visual information provided by the lens(es) of the eyewear worn by the individual. Based upon the settings made in step 910 and in step 920, the physical and sensory program may be initiated in step 930. During the performance of the training program initiated in step 930, sensor data may be collected from wearable sensors describing the performance of the activities during activities the performance of the testing/training program. In step 950 other data collected by external measurement systems may be collected. Based upon the collected data, step 960 may determine whether to adjust the difficulty of the physical and/or sensory components of the training/testing program. If the conclusion of step 960 is that the difficulty should be adjusted, method 900 may proceed to step 970 to increase or decrease the physical and/or sensory difficulty of the program. After step 970, method 900 may then return at the adjusted difficulty level(s) to step 940 to collect sensor data and step 950 to collect other external measurement data with the individual performing the program with increased or decreased physical and/or sensory difficulty. If the outcome of step 960 is that no adjustment of difficulty is required, method 900 may ultimately proceed to step 980 of concluding the testing/training program. Optionally, method 900 may continue to export collected data in step 990, for example through a communication interface to an external computing device.

Method 900 may be performed iteratively for a number of times, either contemporaneously or over the course of hours, days, weeks, months, or even years to provide repeated measurements and/or training of an individual's athletic, sensory, neurological, cognitive, and other functions.

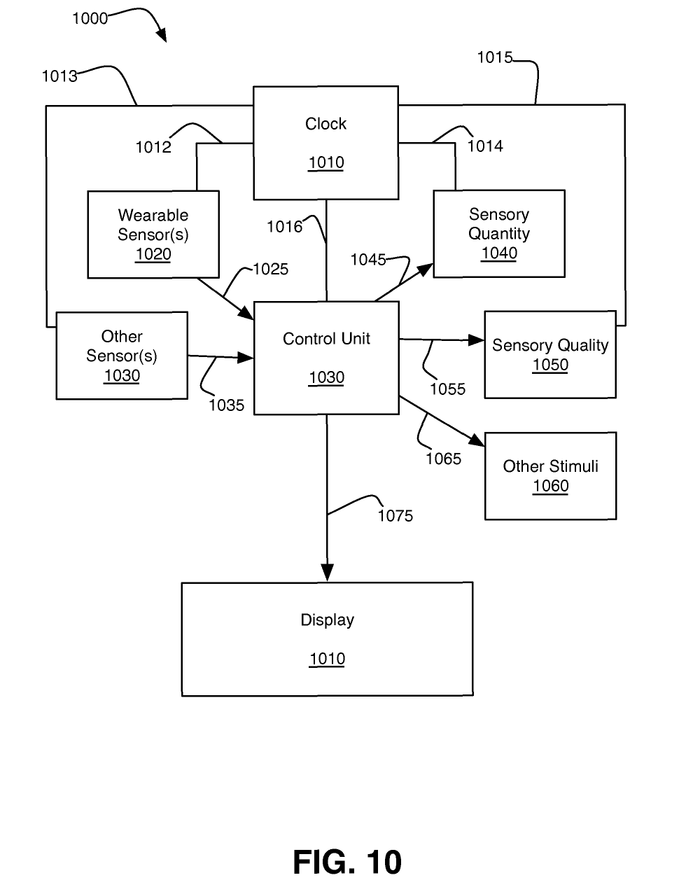

Referring now to FIG. 10, a system 1000 for synchronizing measurements and control of testing/training programs in accordance with the present invention is illustrated. A clock 1010 may provide a common time reference used to calculate the lag involved in reporting measurements made by different types of sensors (or even individual sensor) within system 1000. Sensors may comprise at least wearable sensor(s) 1020 and non-wearable sensor(s) 1030. One or both of wearable sensor(s) 1020 and non-wearable sensor(s) 1030 may comprise further types of sensors and/or individual sensors. For example, wearable sensor(s) 1020 may comprise multiple inertial or other types of sensors, while non-wearable sensor(s) 1030 may comprise one or more optical, infrared, or other position measurement system.

Clock 1010 may communicate 1012 a time to wearable sensor(s) 1020. Clock 1010 may further communicate 1013 a time to non-wearable sensor(s) 1030. A control unit 1030 may also receive 1016 a time from clock 1010. Clock 1010 may directly exchange data with wearable sensor(s) 1020 and/or non-wearable sensor(s) 1030 as shown in the example of FIG. 10, but clock 1010 may alternatively/additionally communicate through a control unit 1030. Additional network elements, media, and/or devices (not shown) may permit clock 1010 to communicate time information as described in the example of FIG. 10.

By associating a time derived from clock 1010 with measurements or other data provided to control unit 1030 by wearable sensor(s) 1020 and non-wearable sensor(s) 1030, and by independently receiving time information from clock 1010 at control unit 1030, the time lag between when a measurement is made and when that measurement is received by control unit 1030 may be measured and accounted for in controlling (via connection 1075) display 1010 to provide symbols directing an individual engaging in a training program, controlling (via connection 1045) sensory quantity 1040 available to the individual, controlling (via connection 1055) sensory quality available to the individual, and/or controlling (via connection 1065) other stimuli 1060 provided to the individual. In some examples, clock 1010 may also communicate 1014 time information to a device (such as eyewear) varying sensory quantity 1040 and may further communicate 1015 time information to a device (such as eyewear) varying sensory quality 1050 in order to provide time information associated with the variance of the quantity and/or quality of visual or other sensory information.

Still referring to FIG. 10, in many examples the same equipment, such as eyewear, may operate to control both the sensory quantity 1040 and sensory quality 1050 provided to an individual, in which case a single communication from clock 1010 and/or a single connection with control unit 1030 may be used. A clock 1010 used in conjunction with the present invention may comprise an external time keeping device or a signal from such an external time keeping device, such as an atomic clock or other device. A signal from such a device may be received/provided over the Internet, by radio, or through other means. Alternatively, clock 1010 may comprise a local device and/or part of a control unit 1030 that provides a suitably consistent indication of the relative time that elapses during the performance of a testing/training program in accordance with the present invention.

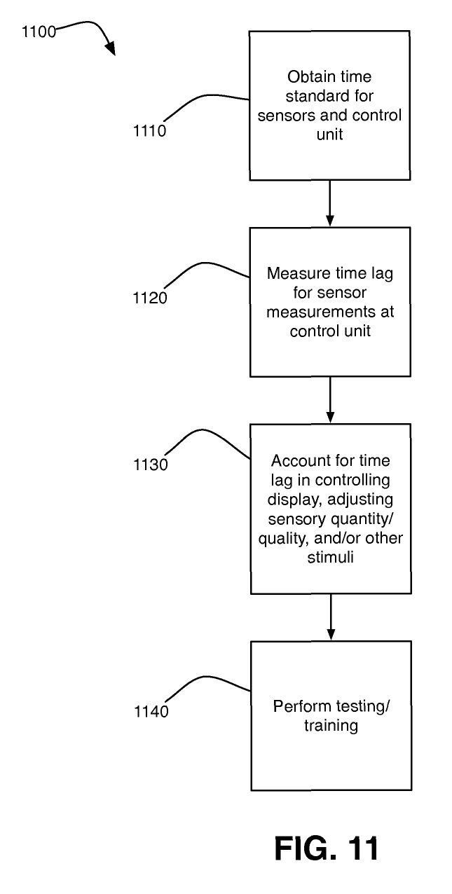

Referring now to FIG. 11, an example of a method 1100 for synchronizing measurements, displays, and or sensory data for a testing/training program in accordance with the present invention is illustrated. In step 1110, time standards may be obtained for at least sensors (such as wearable sensors and non-wearable sensors) and the control unit. Step 1110 may, for example, obtain time standards using a signal received from an external clock, but alternatively may use a time measurement made by control unit. Time standards obtained in step 1110 may, for example, be used to provide a time stamp for measurements made by any type of wearable and/or non-wearable sensor.

Using the time standards obtained in step 1110, the time lag for different types of sensor measurements to arrive at a control unit may be measured in step 1120. For example, by using time stamps associated with measurements received from wearable sensors a first time lag associated with those wearable sensor(s) may be determined, while by using time stamps associated with measurements received from non-wearable sensors a second time lag associated with those non-wearable sensor(s) may be determined. For example, step 1120 may determine that measurements made by wearable sensors require 5 milliseconds to reach the control unit, while measurements made by non-wearable sensors require 15 milliseconds to reach the control unit. These examples of lag are exemplary only, and further method 1100 may be used to account for time lags for individual sensors and/or different types of wearable and/or non-wearable sensors.

In step 1130 the time lag determined in step 1120 may be accounted for in controlling one or more display (for example, to provide instructions to an individual engaging in a testing/training program), in controlling the quantity/quality of sensory data (such as visual information) available to an individual, and/or to control other stimuli provided as part of a testing/training program in accordance with the present invention. Method 1100 may then proceed to step 1140 of providing testing/training to the individual, such as described above. Step 1140 may comprise displaying symbols directing the individual to perform actions as part of a testing/training program, varying the quality and/or quantity of visual information provided to an individual by eyewear in accordance with the present invention, making measurements of an individual's responses using various sensors, etc.

Method 1100 may be performed for each portion of a testing/training program, periodically during a testing/training program, constantly during a testing/training program, or on a predetermined schedule (hourly, daily, weekly, etc.) for equipment to be used as part of a testing/training program.

While the systems and methods of the present invention have been described in examples herein, variation may be made to these examples without departing from the scope of the present invention. More, fewer, and/or different types of sensors than the examples provided herein may be used without departing from the scope of the present invention. The types of training/testing actions described herein may vary considerably from the present examples, and may be particularly related to the rehabilitative and/or athletic training objectives of the associated program for a particular individual. No particular protocol or media for the exchange of information between components of a system in accordance with the present invention is required.

The present invention may be used for any type of physical activity, such as but not limited to athletic training, rehabilitation to improve, restore and/or maintain physical and/or sensory skills that have been or are impaired by injury, illness, and/or age. Such rehabilitation need not be sport related. For any type of training, systems and methods in accordance with the individual may provide the individual training an opportunity to initiate or terminate a training session. The ability to initiate or terminate a training session by the individual training may facilitate the acclimation of sensitive individuals to the training process through frequent but brief training sessions, thereby avoid excessive nausea, vertigo, and similar side effects sometimes encountered as part of perceptual stress training. Further, systems and methods in accordance with the present invention may be helpful in assessing the degree and type of impairment experienced by an individual.

The present invention is not limited to any particular sport or type of training, and may be used for skills, such as basic balance and coordination, that are needed for rehabilitation services. The performance and/or physiological data measured may vary from the examples described herein. In some examples, systems and methods in accordance with the present invention may implement only some types of sensors, such as only performance sensors or only physiological sensors. Similarly, some implementations of the present invention may adjust only the quantity or only the quality of visual information, or may only restrict one of the quality or the quantity of visual information provided.

1. A system for training sensory and physical skills, the system comprising:
eyewear that alters at least the quantity of visual information available to an individual performing a first training task while wearing the eyewear;
at least one sensor that measures at least one performance parameter while the individual performs the first training task; and
at least one control unit that receives the at least one performance parameters from the at least one sensor and causes the eyewear to vary the quantity of visual information available to the individual while the individual performs a second training task while wearing the eyewear.

2. The system for training sensory and physical skills of claim 1, wherein the at least one control unit causes the eyewear to provide a greater quantity of visual information to the individual while the individual performs the second training task if the at least one performance parameter indicates an unsuccessful performance of the first training task and wherein the at least one control unit causes the eyewear to provide a lesser quantity of visual information to the individual while the individual performs the second training task if the at least one performance parameter indicates a successful performance of the first training task.

3. The system for training sensory and physical skills of claim 2, wherein alters both the quantity and the quality of visual information available to the individual performing the first training task, and wherein the at least one control unit further causes the eyewear to provide a greater quality of visual information to the individual while the individual performs the second training task if the at least one performance parameter indicates an unsuccessful performance of the first training task and wherein the at least one control unit causes the eyewear to provide a lesser quality of visual information to the individual while the individual performs the second training task if the at least one performance parameter indicates a successful performance of the first training task.

4. The system for training sensory and physical skills of claim 2, further comprising a display within the eyewear that, under the control of the control unit, displays information to the individual.

5. The system for training sensory and physical skills of claim 4, wherein the information displayed to the individual summarizes the at least one performance parameter.

6. The system for training sensory and physical skills of claim 5, wherein the at least one performance parameter comprises a physiological measurement.

7. The system for training sensory and physical skills of claim 5, wherein the at least one performance parameter comprises a binary indication of the success of the individual at performing the training task.

8. The system for training sensory and physical skills of claim 4, wherein the information displayed to the individual comprises instructions for a subsequent training task to be performed by the individual.

9. A system for training the sensory and physical skills an individual, the system comprising:
eyewear worn by the individual training, the eyewear obscuring at least a portion of the vision of the individual for a first predetermined duration recurring at a first predetermined frequency during training;
at least one physiological sensor that measures a trait of the individual during training;
a control unit receiving measurements from the at least one physiological sensor that causes the eyewear to vary at least one of the duration and the frequency at which the eyewear obscures at least a portion of the vision of the individual in response to the measurements received from the at least one physiological sensor; and
a display component integral to the eyewear that, at the direction of the control unit, displays information regarding the training to the individual.

10. The system for training the sensory and physical skills an individual of claim 9, wherein the at least one physiological sensor measures the center of balance of the individual and wherein the display component displays information describing the center of balance of the individual.

11. The system for training the sensory and physical skills an individual of claim 9, further comprising a result sensor that measures the success of the individual in performing at least one task as part of the training and wherein the control unit receives the measurement of the result sensor.

12. The system for training the sensory and physical skills an individual of claim 11, wherein display component displays information describing the success measured by the result sensor.

13. The system for training the sensory and physical skills an individual of claim 11, wherein the control unit causes the eyewear to vary at least one of the duration and the frequency at which the eyewear obscures at least a portion of the vision of the individual in response to the measurements received from the result sensor.

14. A method for training the sensory and physical skills of an individual, the method comprising:
providing eyewear to the individual to wear while performing a plurality of training tasks, the eyewear operating under the direction of a control unit to vary at least one of the quantity and quality of visual information transmitted through the eyewear to the individual;
using at least one physiological sensor, measuring at least one physiological trait of the individual while the individual performs the plurality of training tasks and transmitting measurements of the at least one physiological trait to the control unit;
using a result sensor, measuring the success of at least some of the training tasks performed by the individual and transmitting the measurements of the result sensor to the control unit;
at the control unit, adjusting at least one of the quantity of visual information transmitted to the individual through the eyewear and the quality of visual information transmitted to the individual through the eyewear based upon at least one of the received measurements of the physiological sensor and the result sensor to consistently challenge the individual; and
under the direction of the control unit, displaying information describing the training to the individual at a display component integral to the eyewear.

15. The method for training the sensory and physical skills of an individual of claim 14, wherein the at least one physiological sensor measures the stability of the individual while performing the plurality of training tasks.

16. The method for training the sensory and physical skills of an individual of claim 15, wherein adjusting at least one of the quantity of visual information transmitted to the individual through the eyewear and the quality of visual information transmitted to the individual through the eyewear based upon at least one of the received measurements of the physiological sensor and the result sensor to consistently challenge the individual comprises increasing at least one of the quantity and quality of visual information transmitted to the individual through the eyewear if the stability of the individual measured by the at least one physiological sensor is less than a predetermined stability threshold and decreasing at least one of the quantity and quality of visual information transmitted to the individual through the eyewear if the stability of the individual measured by the at least one physiological sensor is more than the predetermined stability threshold.

17. The method for training the sensory and physical skills of an individual of claim 16, wherein the predetermined stability threshold comprises a stability of the individual measured by the at least one physiological sensor during a prior plurality of training tasks performed by the individual.

18. The method for training the sensory and physical skills of an individual of claim 14, wherein adjusting at least one of the quantity of visual information transmitted to the individual through the eyewear and the quality of visual information transmitted to the individual through the eyewear based upon at least one of the received measurements of the physiological sensor and the result sensor to consistently challenge the individual comprises increasing at least one of the quantity and quality of visual information transmitted to the individual through the eyewear if the result sensor measures the success of the individual in performing the plurality of training tasks has less than a predetermined success threshold and decreasing at least one of the quantity and quality of visual information transmitted to the individual through the eyewear if the result sensor measures the success of the individual in performing the plurality of training tasks as more than a predetermined stability threshold.

19. The method for training the sensory and physical skills of an individual of claim 14, further comprising, at the control unit, adjusting a difficulty of the plurality of training tasks performed by the individual based upon at least one of the received measurements of the physiological sensor and the result sensor to consistently challenge the individual and communicating the adjustment of the difficulty of the plurality of training tasks to the individual using the display component.

20. The method for training the sensory and physical skills of an individual of claim 14, wherein the plurality of training tasks comprise different physical tasks.
